(19) United States
(12) Patent Application Publication
(10) Pub. No.: US 2026/0360513 A1
(43) Pub. Date: Nov. 12, 2026

(54) NATIVE CLOUD TIERING USING CLINICALLY INTELLIGENT IMAGE LIFECYCLE MANAGEMENT

(71) Applicant: FUJIFILM HEALTHCARE AMERICAS CORPORATION, Lexington, MA (US)

(72) Inventors: John Hansen, Shawnee, KS (US); Doug Stelpflug, Wauwatosa, WI (US); Chris Baumgartner, Wauwatosa, WI (US)

(73) Assignee: FUJIFILM HEALTHCARE AMERICAS CORPORATION, Lexington, MA (US)

(21) Appl. No.: 19/200,469

(22) Filed: May 6, 2025

(51) Int. Cl. G16H 30/20; G16H 80/00

(52) U.S. Cl. CPC G16H 30/20; G16H 80/00

(57) ABSTRACT

An image lifecycle management system for handling medical images in a healthcare enterprise network includes an interface processor, a database system with temporary and long-term storage components, and an archive managing processor. The system analyzes clinical metadata using clinical intelligent rules to classify medical image data and assigns the classified data to Storage Management Organization (STMO) processors. Each STMO processor defines access permissions, cloud storage placement, and lifecycle management for its assigned data. The system connects to cloud storage services via an Application Programming Interface (API), with storage organized in hierarchical tiers offering different cost and performance characteristics.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright or rights whatsoever.

### INCORPORATION BY REFERENCE

The entire disclosure of U.S. Patent No. 7,583,861, issued Sep. 1, 2009, is incorporated by reference for all purposes.

### FIELD OF THE INVENTION

The disclosed subject matter relates to the field of medical imaging, and, more specifically, to systems for storing and managing medical image data for a healthcare enterprise and methods thereof.

### BACKGROUND

Medical images and associated medical data are acquired by a large number of different imaging modalities, including X-ray imaging, computed tomography (CT), radioisotope emission imaging, computed emission tomography, magnetic resonance imaging, ultrasonic imaging, and numerous other specialized imaging systems. These digital data must be stored, retrieved, and managed efficiently within healthcare enterprises.

Traditional computer systems for storage, retrieval, and viewing of these digital data (referred to as Picture Archiving and Communication, “PAC systems,” or “PACS”), though quite satisfactory for use throughout a single department in a hospital, typically have limited amounts of digital storage and thus lack the capability of managing these digital data from a large healthcare enterprise indefinitely. This further creates a substantial bottleneck, as imaging technology continues to advance, and volumes of medical images grow exponentially. Therefore, there is an increasing demand for cloud-based storage solutions.

Current major cloud-based storage providers offer virtually unlimited storage capacity, multiple storage tiers, and support for various data operations, including e.g., ingress (initial storage), ongoing storage, and egress (data retrieval). The various tiers can have different pricing for the data operations. For example, a first tier can have relatively expensive storage, but inexpensive ingress and egress. Such a tier can be beneficial for data that will need to be accessed often. A second tier can have relatively inexpensive storage, but more expensive ingress and egress. Such a tier can be beneficial for data that will be accessed infrequently. Accordingly, these providers enable healthcare enterprises to optimize their data management based on cost and access requirements.

However, a significant limitation of current cloud-based storage solutions is the lack of clinical intelligence in the rules for migrating image data across storage tiers. Existing approaches typically rely on system-wide “time since last access” rules. For example, the systems can automatically migrate image data to less expensive storage tiers if the data has not been accessed for a certain amount of time (e.g., 1 year or 5 years). The same rules can be applied to all image data regardless of its clinical context. This one-size-fits-all approach fails to recognize that different types of medical images have different lifecycle patterns and access needs. Additionally, it can cause unnecessary costs from migration of unused image data, which can be avoided by initially designating the image data to appropriate storage tiers.

For example, point-of-care ultrasound (POCUS) image data acquired from emergency departments is rarely accessed after initial acquisition and interpretation, making POCUS image data ideal candidates for immediate placement in lower-cost storage tiers rather than waiting for system-wide time-based rules to trigger migration. Mammography images, unlike general radiology data, which are generally moved to lower-cost storage tiers quickly, may need to be readily available for comparison over many years, and thus, can be maintained in cloud storage tiers with lower retrieval latency and lower egress cost over extended periods than general radiology data. As another example, wound care images can become less clinically relevant once a patient's condition is resolved, while certain cardiovascular data, which can involve echocardiogram and cardiac angiography studies that require periodic review during ongoing cardiac treatment, also benefit from extended periods of storage with lower ingress/egress cost. Similarly, pathology images, particularly for oncology patients, can need variable accessibility patterns aligned with treatment protocols and follow-up schedules. Current systems do not differentiate between these varied clinical use cases when determining storage tiers.

Furthermore, in a complex data environment, the sole reliance on system-wide time-based rules applied to various clinical contexts, without incorporating clinically intelligent rules, can make the situation worse. For example, healthcare enterprises commonly generate and manage both standardized data, such as Digital Imaging and Communications in Medicine (DICOM) data, and non-DICOM data, including JPEG images, MPEG videos, PDFs, and other native file formats. In particular, existing approaches typically lack integrated lifecycle management for non-DICOM data, despite the growing importance of such data in modern healthcare. As a result, cloud-based storage providers may be unable to store and assign non-DICOM data to specific tiers within their storage systems, or are limited to only performing basic operations like deletion, rather than intelligent migration between storage tiers.

### SUMMARY OF THE INVENTION

The disclosed subject matter addresses these limitations by providing a clinically intelligent image lifecycle management system that applies domain-specific rules to optimize the storage of both DICOM and non-DICOM medical images across multiple cloud storage tiers.

The disclosed subject matter relates to an image lifecycle management system connected to receive messages over a healthcare enterprise network. The system can include an interface processor for receiving and forwarding data comprising medical image data and associated clinical metadata. The system can include a database system comprising a first storage medium for temporary storage and a solid-state drive for long-term storage of metadata.

In accordance with the disclosed subject matter, the system can include an archive managing processor configured to temporarily store the data forwarded from the interface processor in the fast storage medium and analyze the metadata based on clinically intelligent rules and classify the image data based on the analyzed metadata. The archive managing processor can assign the classified image data to a plurality of designated Storage Management Organization (STMO) processors, respectively. Each STMO processor can execute a command message on its respective image data, defining access control permissions, initial cloud storage tier placement of its respective data, and automated lifecycle management.

The system includes a storage interface processor for connecting to the cloud storage services via an application programming interface (API), wherein a plurality of storage devices are organized in tiers, and each tier has cost and performance characteristics that differ from other tiers. In some embodiments, the system includes means for transferring the respective image data stored in the fast storage medium to the respective cloud storage tier, wherein the transferring is responsive to the respective command message executed by each STMO processor.

The archive managing processor is further configured to separate the data into the medical image data and the clinical metadata, wherein the clinical metadata is transferred and stored in the solid-state drive. The data comprising medical image data and associated clinical metadata is received from medical imaging devices on the healthcare enterprise network.

The access control permissions determine which users and systems can view, modify, or delete the respective classified data of each STMO processor. The initial cloud storage tier placement can be based on anticipated clinical relevance and access frequency as determined by the clinically intelligent rules. The automated lifecycle management can determine migration of respective medical image data between cloud storage tiers as clinical relevance changes over time in accordance with organizational image retention policies.

In accordance with the disclosed subject matter, the system manages both Digital Imaging and Communications in Medicine (DICOM) and non-DICOM data. The non-DICOM data comprises Joint Photographic Experts Group (JPEG) images, Portable Network Graphics (PNG) images, Tagged Image File Format (TIFF) images, Portable Document Format (PDF) files, Moving Picture Experts Group (MPEG) videos, Audio Video Interleave (AVI) videos and other medical imaging and documentation formats.

The migration of image data between storage tiers is performed using API calls to reclassify the image data within the cloud storage service provider in place without physically retrieving and re-storing the image data.

### BRIEF DESCRIPTION OF THE DRAWINGS

In the drawings:

FIG. 1 illustrates an architecture for a system for intelligent image lifecycle management system, in accordance with the disclosed subject matter.

FIG. 2 is a block diagram of an exemplary architecture of the image lifecycle management system of FIG. 1.

FIG. 3 is a block diagram of an exemplary architecture of the cloud storage service provider that communicates with the image lifecycle management system, as illustrated in FIG. 1.

FIG. 4 is a block diagram showing an example of computer architecture for a device capable of executing program components for implementing the abovementioned functionality.

### DETAILED DESCRIPTION OF THE PREFERRED EMBODIMENT

In the following description, for the purposes of explanation, numerous specific details are set forth in order to provide a thorough understanding of the present invention. It will be apparent, however, that the present invention can be practiced without these specific details. In other instances, well-known structures and devices are shown in block diagram form in order to avoid unnecessarily obscuring the present invention.

The text of this disclosure, in combination with the drawing figures, is intended to describe in prose various embodiments of the disclosed technology, at the same level of detail that is used by people of skill in the arts to which this disclosure pertains to communicate with one another. That is, the level of detail set forth in this disclosure is the same level of detail that persons of skill in the art normally use to communicate with one another to implement the inventions claimed herein.

### 1. General Overview

Reference will now be made in detail to various exemplary embodiments of the disclosed subject matter, exemplary embodiments of which are illustrated in the accompanying drawings. As used in the description and the appended claims, the singular forms, such as “a,” “an,” “the,” and singular nouns, are intended to include the plural forms as well, unless the context clearly indicates otherwise.

In accordance with the disclosed subject matter, systems and methods for managing medical data are provided. For example, a method of clinically intelligent image lifecycle management can include receiving medical image data, analyzing clinical metadata associated with the image data, applying clinically relevant classification rules, and implementing automated storage tier assignment and migration strategies based on the clinical classification.

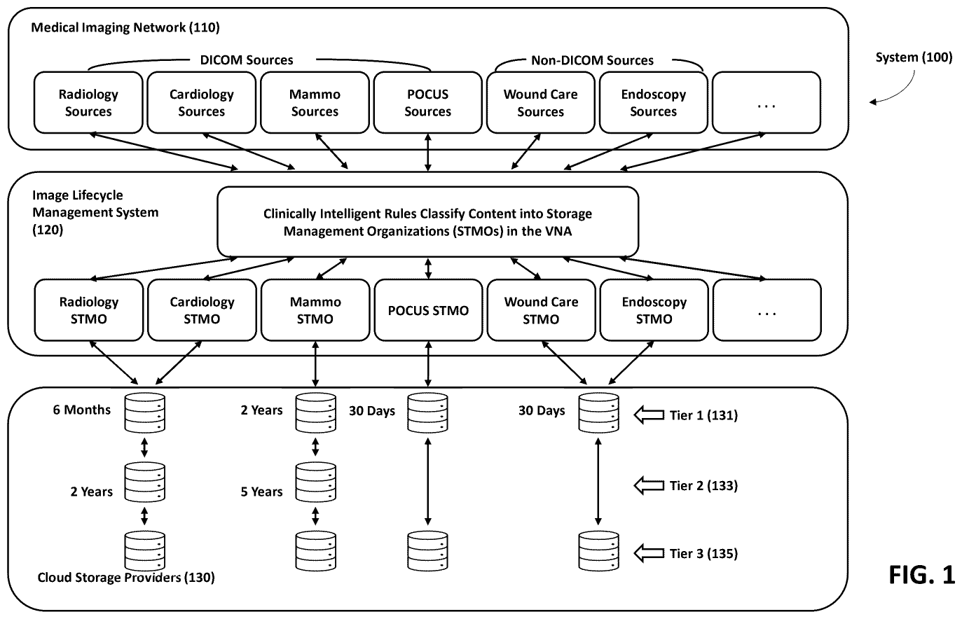

The disclosed subject matter can include systems and methods for optimizing cloud storage of medical images through clinically intelligent lifecycle management. For the purpose of illustration and not limitation, the fundamental structure of a clinically intelligent image lifecycle management system is constructed with reference to FIG. 1.

FIG. 1 illustrates the architecture for a system (100) for storing and managing medical image data for a healthcare enterprise. The illustrated system includes a medical imaging network (110) that connects various input sources of medical images throughout the healthcare enterprise, a clinically intelligent image lifecycle management system (120), and connections to multiple cloud storage providers (130) offering tiered storage options.

The medical imaging network (110) encompasses the diverse array of imaging devices and information systems that generate and initially process medical image data. This includes traditional DICOM-generating modalities such as radiographic sources (such as X-ray machines, CT scanners), cardiovascular sources (such as cardiac catheterization laboratories, electrocardiograms and echocardiography systems), mammography sources (such as screening and diagnostic mammogram machines), and ultrasound sources (such as POCUS devices and diagnostic ultrasound systems). Other exemplary DICOM sources can include nuclear medicine sources, magnetic resonance sources, digital pathology sources, radiotherapy sources and other modality types. The medical imaging network (110) can also include non-DICOM image sources including dermatology sources, wound care sources (such as wound measurement systems, digital cameras, thermal imaging devices), and endoscopy sources (such as gastrointestinal endoscopes, bronchoscopes, colonoscopes). Other exemplary non-DICOM image sources can include point-of-care cameras, digital video recorders, mobile devices, and specialty department systems such as arthroscopes, otoscopes, and others.

The network (110) also incorporates the various PACS, Radiology Information systems (RIS), specialized department systems that manage these images within the healthcare enterprise, or institution-scaled Hospital Information systems (HIS). PACS are specialized computer systems dedicated to storing, retrieving, distributing, and presenting medical images, providing digital alternatives to traditional film-based image management for radiology departments. These systems comprise imaging devices (modalities), secure networks for image transmission, workstations for viewing images, and archives for image storage. Like PACS, RISs are usually designed as a client-server system or web application that requires particular software to manage radiological data and workflow. The HIS is a comprehensive, integrated information system for managing patient financial and billing, admissions, discharge, and transfer. Together, these three systems form the core information infrastructure of modern healthcare enterprises in which medical imaging is a byproduct of the care process and where the clinically intelligent image lifecycle management system can enhance their data flow capabilities.

The clinically intelligent image lifecycle management system (120) can serve as the central interface between the medical imaging network (110) of a health enterprise and cloud storage service providers (130). This system (120) analyzes incoming medical images, applies clinically relevant rules, and directs images to appropriate storage tiers. Unlike traditional storage management systems that rely solely on system-wide time-based metrics, this system incorporates domain-specific knowledge about different types of medical images and their clinical usage patterns.

The cloud storage providers (130) further offer multiple storage tiers with varying costs and performance characteristics. These typically include high-performance, relatively more expensive, immediate-access tiers (131) for frequently accessed data, intermediate, relatively mid-level expensive tiers (133) for less frequently accessed data, and archival, relatively inexpensive tiers (135) for rarely accessed data. The clinically intelligent image lifecycle management system (120) interacts with these providers through standardized APIs to implement optimal storage strategies. These standardized APIs are programming interfaces that define structured methods for various system applications to communicate with cloud storage service providers, allowing operations such as object storage, retrieval, deletion, tier management, and metadata manipulation with consistent protocols.

### 2. Structural and Functional Overview

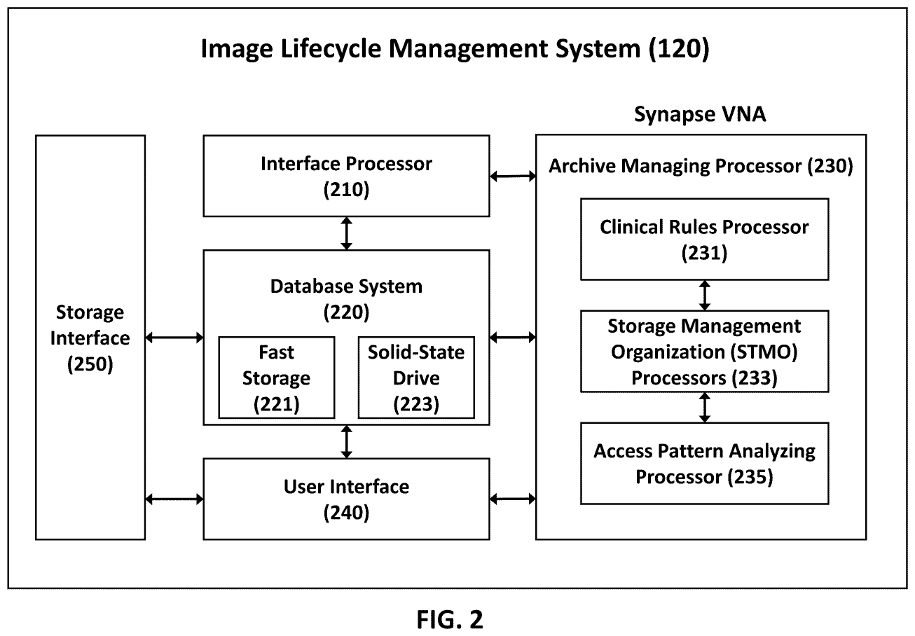

The disclosed subject matter can be implemented on standard computing hardware with appropriate software modifications to implement clinically intelligent storage management. FIG. 2 illustrates an exemplary architecture of the image lifecycle management system (120) shown in FIG. 1 for storing and managing medical image data for a healthcare enterprise. It provides a schematic overview of the hardware and software components involved in implementing the clinically intelligent storage management system. The system includes an interface processor (210), a database system (220), an archive managing processor (230), a user interface (240), and a storage interface processor (250).

### 2.1 Interface Processor

The interface processor (210) shown in FIG. 2 serves as the entry point for medical images from various sources throughout the healthcare enterprise. The interface processor (210) can receive standardized Digital Imaging and Communications in Medicine (DICOM) images and extract relevant metadata for clinical classification. Additionally, the interface processor (210) can handle non-DICOM data and processes various image formats, including Joint Photographic Experts Group (JPEG), Portable Network Graphics (PNG), Tagged Image File Format (TIFF), Portable Document Format (PDF), and video formats such as Moving Picture Experts Group (MPEG) and Audio Video Interleave (AVI) and other medical imaging and documentation formats.

In accordance with the disclosed subject matter, the interface processor (210) can extract clinical metadata from both DICOM and non-DICOM sources, and normalize this information (for example, adjusting the resolution of the images or size of the files) for consistent processing. For DICOM images, the processor can extract comprehensive metadata from the DICOM header, including patient demographics (name, identification number, date of birth, gender), study information (accession number, study date, referring physician), series information (modality, series description, body part examined), and image acquisition parameters (specific to each modality).

Here provided is an exemplary list of DICOM metadata available to the processor: Accession Number, Acquisition Date, Acquisition Time, Body Part, Content Date, Content Time, Ethnic Group, Image Number, Image Type, Institution Name, Institutional Department Name, Issuer of Patient ID, Laterality, Modality, Patient's Age, Patient's Birth Date/Time, Patient's First Name, Patient's Last Name, Patient's Sex, Patient's Size, Patient's Weight, Patient ID, Procedure Code Value, Referring Physician's First Name, Referring Physician's Last Name, Requesting Physician's First Name, Requesting Physician's Last Name, Requesting Service, SOP Class UID, SOP Instance UID, Scheduled Study Start Date/Time, Series Description, Series Number, Series UID, Slice Thickness, Station Name, Study Completion Date/Time, Study Date/Time, Study Description, Study ID. Metadata can also be inferred from the source systems that send images into the processor. The processor can select any single metadata field or combine multiple fields to create clinically relevant rules, tailoring the system to their specific storage management needs and clinical workflows.

For non-DICOM images, the processor (210) can extract embedded metadata such as Exchangeable Image File Format (EXIF) data and timestamps, parse text information associated with documents and images, normalize terminology across different sources and systems, and map proprietary metadata fields to standard classifications. Additional metadata can be applied to non-DICOM files uploaded manually via the user interface. The metadata model is open and configurable such that certain types of non-DICOM files can have different associated metadata compared to other non-DICOM file types.

The interface processor (210) can further directly communicate with other hospital information systems to gather additional clinical context for images. This includes integration with Electronic Health Record (EHR) systems to access patient diagnoses, connections to RIS for study details, links to specialty department systems (endoscopy, electrocardiogramdiology, pathology, dermatology), and scheduling system integration to identify active and resolved cases.

### 2.2 Database System

The database system (220) shown in FIG. 2 can include two primary components: a fast storage medium (221) for temporary storage of image data, and a solid-state drive (223) for long-term storage of metadata. In some embodiments, the image data stored in the fast medium will ultimately be transferred to cloud storage providers without being processed or transferred within the system. The metadata, however, remains within the database system (220) and can be modified and retrieved to control the image data in the cloud providers.

The fast storage medium (221) can be implemented using high-performance solid-state drives (223) arranged in a Redundant Array of Independent Disks (RAID) configuration to provide both speed and reliability. This temporary storage can serve as a buffer between incoming medical images and their ultimate cloud storage destination, allowing the system to receive large volumes of image data without delay, regardless of network conditions to the cloud storage providers. The system can utilize non-volatile memory express (NVMe) technology for this temporary storage to achieve transfer rates of multiple gigabytes per second, ensuring that even high-resolution imaging studies can be ingested rapidly.

The metadata storage component can utilize enterprise-grade solid-state drives (223) optimized for database operations with high input/output operations per second capabilities. This storage component maintains a comprehensive relational database that contains detailed information about each stored image, including patient information, study details, clinical context, and cloud storage locations. The database schema is specifically designed to support rapid queries based on any combination of clinical, administrative, or technical parameters, enabling efficient retrieval of specific images or cohorts of images based on clinical need.

The database system (220) implements advanced caching mechanisms to optimize performance. Frequently accessed metadata is maintained in system memory for immediate access, while less frequently accessed information remains on the solid-state drives (223). Additionally, the database system can employ database sharing techniques for serving extremely large healthcare enterprises, distributing metadata across multiple physical storage devices while maintaining logical consistency and accessibility.

The database system (220) can maintain a registry that maps each stored image to its current location within the cloud storage tiers. This registry can be updated whenever images are migrated between tiers, ensuring that the system can always locate any stored image regardless of its current storage tier. The registry can also record the complete migration history of each image, providing an audit trail for compliance purposes and data for continual refinement of the clinical rules that govern storage decisions.

The database system (220) can incorporate backup and recovery mechanisms to ensure the safety and availability of metadata. These mechanisms can include real-time replication to geographically separated backup systems, point-in-time recovery capabilities, and automated safety verification. Since the metadata serves as the key to locating and managing all stored images, its protection is critical to the overall reliability of the system.

### 2.3 Archive Managing Processor

The archive managing processor (230), labeled in FIG. 2 as part of the Synapse Vendor Neutral Archive (Synapse VNA), implements the core intelligence of the system. It analyzes clinical metadata and applies appropriate rules to determine optimal storage strategies. As shown in FIG. 2, the archive managing processor (230) includes a clinical rules processor (231), multiple Storage Management Organization (STMO) processors (233), and an access pattern analyzing processor (235).

The clinical rules processor (231) can contain domain-specific knowledge about different types of medical images and their typical lifecycle patterns. For example, the domains can include specialty-specific rule sets for radiology, cardiology, pathology, dermatology, wound care, and other clinical domains; modality-specific rules for different imaging technologies (Magnetic Resonance Imaging, CT, ultrasound, etc.); anatomic region classifiers that identify body parts being imaged; clinical context analyzers that consider patient diagnoses; and image purpose determinants that distinguish between diagnostic, screening, and monitoring studies.

The clinical rules processor (231) can apply a multi-step process to determine appropriate storage strategies. First, it can categorize the image by identifying the modality (for example, Magnetic Resonance Imaging, CT, ultrasound, photograph). Following categorization can be based on determining the clinical specialty (for example, radiology, cardiology, dermatology), classifying the anatomical region (for example, brain, heart, skin), and recognizing the image purpose (for example, diagnosis, screening, monitoring). This multi-dimensional categorization enables the application of highly specific clinical rules for storage strategy.

The clinical rules processor (231) can also analyze the clinical context by examining patient diagnosis and condition (for example, acute, chronic, resolved), treatment status (for example, active treatment, follow-up, completed), related procedures and events (for example, surgery, chemotherapy), and provider specialties involved in the patient's care. This analysis places the image in its broader clinical context, which can significantly influence its expected access pattern.

The Storage Management Organization (STMO) processors (233) shown in FIG. 2 can determine when and where to migrate images based on the clinically intelligent rules configured and associated with the STMOs. The STMO processors (233) can also have multiple customized image lifecycle management (ILM) rules that govern the migration plans and optimal transition points between storage tiers, plan for batch migrations to minimize operational overhead, and schedule migrations during low-activity periods.

As illustrated in FIG. 2, both the clinical rules processor (231, enabling the clinical rules) and the STMO processors (233, enabling the ILM rules) can operate on the DICOM metadata and non-DICOM files that are available through the connection to the database system (220) to make clinically relevant and intelligent storage decisions. The clinical rules processor (231) can first analyze the exemplary DICOM metadata fields to classify medical images based on their clinical context. For example, by examining DICOM fields such as Modality, Body Part, and Study Description, the processor (231) can classify a mammography screening study as requiring long-term comparative access. Similarly, by analyzing the Requesting Service and Institution Department Name fields, the processor (231) can identify emergency department POCUS images that rarely need retrieval after initial diagnosis.

Based on these clinical classifications, the archive managing processor (230) assigns the classified image data to one or more designated STMO processors (233). Each STMO processor (233) then executes a command message that uses the same DICOM metadata to generate ILM rules to determine initial cloud storage placement. For example, one STMO processor can direct emergency POCUS images to low-cost storage while maintaining screening mammograms in high-performance storage by establishing migration timelines based on Acquisition Date and clinical classification. Meanwhile, a different STMO processor can organize batch migrations using Institution Name and Study ID for efficiency, or clinical factors like Body Part and Procedure Code with expected retrieval needs.

This integrated configuration can ensure that storage decisions are driven by clinical relevance rather than system-wide time-based rules, optimizing both cost and accessibility throughout the image lifecycle. Although all the listed DICOM metadata fields and non-DICOM files are available through the database system (220), the clinical rules processor (231) and STMO processors (233) can use different subsets of these data for their respective functions. For example, the clinical rule processors (231) and one or more of the STMO processors (233) can use different DICOM metadata fields or different non-DICOM files for their specific clinical rules or ILM rules. Additionally, for analyzing and managing the same image data, one or more of the STMO processors can use different DICOM metadata fields or different non-DICOM files for their specific ILM rules determination than those used by the clinical rules processor (231).

The access pattern analyzing processor (235) shown in FIG. 2 can study historical access patterns to inform storage decisions. These functions include frequency analysis that tracks how often different image types are accessed, temporal pattern recognition that identifies trends in access timing, user and role analysis that examines which healthcare providers access different image types, correlation with clinical events that links image access to patient encounters and treatments, and predictive modeling that forecasts future access needs based on historical patterns. The access pattern analyzing processor (235) can also ensure all storage decisions align with regulatory requirements and organizational policies through retention policy enforcement, privacy rule implementation, legal hold processing, and regional compliance, adapting to different jurisdictional requirements.

### 2.4 User Interface

The user interface (240) shown in FIG. 2 allows administrators to configure and monitor the system. It enables administrators to define and modify clinical storage rules and STMO rules, displays real-time metrics on system performance and storage utilization, and allows administrators to manually adjust storage decisions when necessary. The user interface (240) can control and determine all the processors and processes.

### 2.5 Storage Interface Processor

The storage interface processor (250) shown in FIG. 2 can manage communications with cloud storage providers. The storage interface processor (250) can provide standardized interfaces to different cloud storage providers through Application Programming Interface (API) management, requests cloud storage providers to migrate data between storage tiers, tracks data transfer rates, latency, and availability metrics, and requests encryption and security management functions to ensure data security during storage and transfer. In case of error migration. The storage interface processor (250) can request providers to check failure scenarios and implement appropriate recovery mechanisms.

Overall, the image lifecycle management system (120) can interconnect each component by bidirectional data flow paths that facilitate the transfer of data and control signals. Primary data flow paths can transmit the files to be stored by connecting the interface processor (210) to the archive managing processor (230), and the database system (220) to store temporarily transmitted files to be transferred to the cloud later. Secondary data flow paths enable communication between the processors of the Synapse VNA. For example, the clinical rules processor (231) will send the first classification results to the STMO processors (233) for further analysis. The STMO processors (233) will later generate commands based on the second classification for the access pattern analyzing processor (235) to designate the access control, the lifecycle storage of the data, and the location (the storage tiers) in the cloud.

The archive managing processor (230) can execute operations based on categorized request types that define the nature of the action to be performed. These request categories serve as a classification system for managing the complete lifecycle of medical image data. The archive managing processor (230) can recognize and process at least four fundamental request categories: a storage category, a retrieve category, a delete category, and a migrate category. The storage category encompasses requests to initially store medical image data in designated cloud storage tiers when new images are acquired from imaging devices. The retrieve category includes requests to access previously stored image data when needed for clinical review, comparison, or other healthcare activities. The migrate category comprises requests to reclassify image data between different storage tiers based on clinical relevance as determined by the clinical rules processor (231). Each STMO processor (233) generates command messages associated with these specific categories, enabling the system to efficiently manage different stages of the medical image lifecycle through appropriate API interactions with the cloud storage service providers.

### 3. Cloud Storage Provider Integration

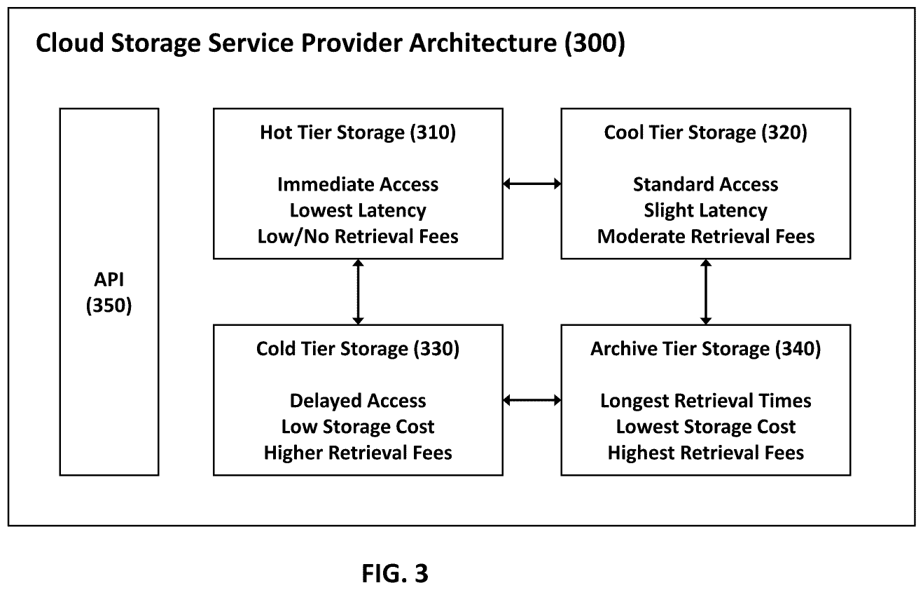

FIG. 3 illustrates a cloud storage service provider (300) that communicates with the image lifecycle management system. While specific implementations vary between providers, most cloud storage platforms offer similar capabilities through different interfaces. The system includes an Application Programming Interface (API) (350) that receives commands from the storage interface processor (250) from the imaging management system shown in FIG. 2.

### 3.1 Storage Tiers

Cloud storage providers typically offer multiple storage tiers with different performance characteristics and cost structures, as shown in FIG. 3. The tiers can include the hot tier storage (310), which provides immediate access with the lowest latency and high throughput. This tier has the highest storage cost but minimal retrieval fees. The cool tier storage (320) offers standard access with slight latency at a lower storage cost, with moderate retrieval fees. The cold tier storage (330) provides slower access at a low storage cost but with higher retrieval fees. The archive tier storage (340) offers the lowest storage costs but with the longest retrieval times and the highest retrieval fees.

These tiers allow for cost optimization based on access frequency and retrieval urgency. For example, POCUS images can initially reside in hot tier storage (310) while a patient is in the emergency department, but can be quickly migrated to tiers with lower storage costs, such as cool tier storage (320) or cold tier storage (330), within days or weeks of the encounter, as these images rarely need subsequent review after the initial episode of care. Conversely, mammography studies can remain clinically relevant for longitudinal comparison over many years, justifying their extended residence in more accessible tiers, such as the hot tier (310), despite higher ongoing storage costs.

On the other hand, since cold and archive tier storage offer significantly reduced storage costs, higher retrieval fees, and longer access times, they are appropriate for images with declining clinical relevance. General radiology studies like X-rays of broken bones can transition to these tiers after the initial treatment phase ends, which typically occurs within months of acquisition.

Furthermore, the clinically intelligent image lifecycle management system can also consider both image type and patient-specific factors when determining optimal tier placement. For example, imaging studies for chronic conditions or oncology follow-up may warrant different migration paths than those for acute conditions, with the system capable of adjusting these parameters based on diagnosis codes, treatment protocols, and historical access patterns specific to each healthcare organization's clinical practices.

### 3.2 Data Operations

As illustrated in FIG. 3, the cloud storage service provider architecture (300) implements bidirectional communication pathways between the various storage tiers. These bidirectional connections, represented by the two-headed arrows between storage tiers, enable the free migration of medical image data throughout its lifecycle without requiring intermediate steps. The connectivity within all tiers allows for direct data reclassification between any combination of tiers based on clinical requirements. This direct migration capability, facilitated through the API (350) command from the clinically intelligent image lifecycle management system, significantly reduces operational overhead and minimizes transaction costs by eliminating the need to physically move the data when changing storage classifications between tiers.

Through these bidirectional pathways, the system can effectively classify content to the appropriate tier so that the least accessed data, based on clinically intelligent rules, is migrated to the lowest cost tier as quickly as possible. For example, cloud storage service providers can support various data operations related to storing and retrieving medical images. Ingress operations involve uploading new data to the cloud storage platform and are typically free or low-cost. Storage operations maintain data within a particular storage tier, with costs usually calculated per gigabyte per month. Egress operations retrieve data from storage, with costs varying significantly based on the storage tier and data volume. Migration operations move data between storage tiers and can incur both retrieval and ingress costs. Management operations involve API calls to list, describe, or manipulate stored data objects, and typically incur minimal costs, but can add up with high-volume operations.

### 3.3 API and Integration

Cloud providers expose their services through standardized APIs (350), shown in FIG. 3, that allow the image lifecycle management system to interact with their storage platforms. These include APIs that can provide web-based interfaces for storage operations, libraries for various programming languages that simplify integration, security and authentication mechanisms to ensure secure access, rate limiting and throttling controls to manage resource utilization, and notification services that alert the lifecycle management system to events and state changes. The storage interface processor (250) shown in FIG. 2 uses these APIs to implement the storage decisions made by the archive managing processor (230).

These standardized APIs can include Representational State Transfer (REST) interfaces that enable programmatic control over storage operations through HTTP-based commands, Data Object APIs that provide methods for storing, retrieving, and manipulating discrete data objects, and Tier Management APIs that specifically facilitate the reclassification of stored objects between different storage tiers without physically transferring the data. Additionally, the system can utilize Software Development Kit (SDK) libraries provided by the cloud storage service providers to simplify these API interactions and ensure reliable communication between the image management system and the cloud storage service infrastructure.

### 4. System Operation

During operation, the interface processor (210) can receive medical images from various sources and extract relevant metadata. The archive managing processor (230) analyzes this metadata using clinical rules to determine the optimal storage strategy. The storage interface processor (250) then implements this strategy by placing the image in the appropriate cloud storage tier. The system continuously monitors access patterns and clinical factors to optimize storage decisions over time.

One particular advantage of the disclosed system is its ability to optimize cloud storage costs by applying clinically relevant migration rules. Unlike traditional systems that use the same simplistic system-wide time-based rules that apply to all data for tier migration, the disclosed system considers the clinical context and expected access patterns of different image types. For example, POCUS images from emergency departments can be moved to cold storage tiers more rapidly than mammography images, which typically require periodic review over many years.

The system can leverage cloud storage tier reclassification capabilities to migrate data between tiers without physically moving or transferring the data, thereby avoiding more costly data transaction fees. When a customized IML rule determines it is time to move content from one cloud storage tier to another, the system can make an API call to the cloud storage provider to reclassify the content in place, rather than retrieving the data from one tier, storing the data to a new tier, and deleting the data from the original tier.

Examples of clinically intelligent storage rules include POCUS data from emergency departments which can initially be stored in hot tier storage (310) for immediate access during the patient encounter, then migrated to cool tier storage (320) after 14 days, and finally to cold tier storage (330) after 60 days, as these images are rarely accessed after the initial diagnosis. General radiology images can be stored in hot tier storage (310) for 90 days to support active treatment and initial follow-up, then migrated to cool tier storage (320) for one year to support periodic review, and finally to cold tier storage (330) for long-term retention. Mammography images, due to their critical role in longitudinal comparison, can remain in hot tier storage (310) for one year, then move to cool tier storage (320) for an additional four years to support regular screening comparisons, before transitioning to cold tier storage (330). Wound care images can follow an episode-based lifecycle, rapidly transitioning through cool tier storage (320) to cold tier storage (330) on a different schedule than other types of images based on clinically relevant rules determined by the health system.

Each of these examples demonstrates how the system applies domain-specific clinical knowledge to storage decisions, resulting in optimized storage costs without compromising clinical care.

### 5. Hardware Infrastructure

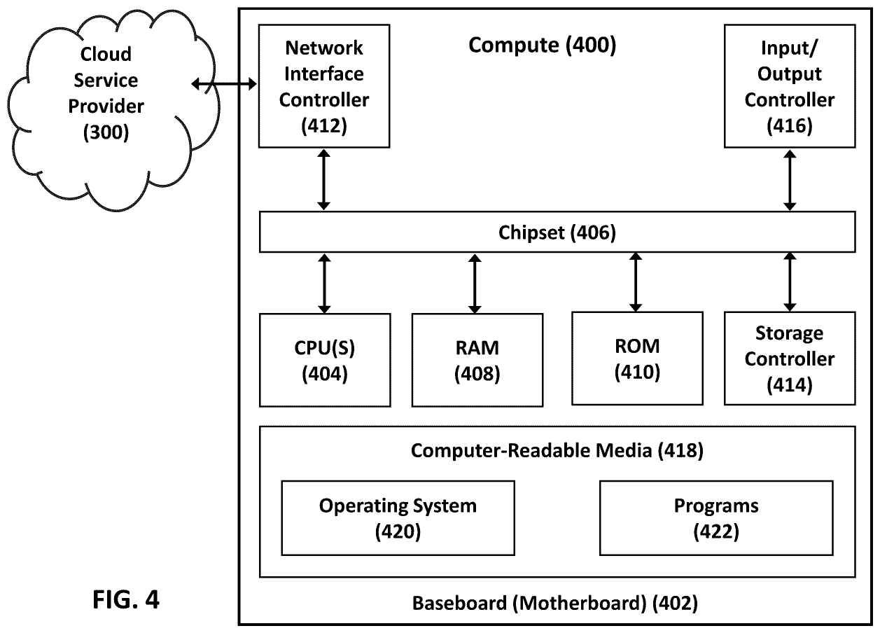

FIG. 4 illustrates a block diagram showing an example of computer architecture for a device capable of executing program components for implementing the abovementioned functionality. The computer architecture, as shown in FIG. 4, illustrates a computing device (400) that can be utilized to execute any of the software components presented herein.

The computing device (400) can correspond to any hardware component of the image lifecycle management system (120) shown in FIG. 2, including the interface processor (210), the database system (220), the archive managing processor (230), and the storage interface processor (250). The computing device (400) can be implemented as a server computer, workstation, desktop computer, or other computing device suitable for enterprise-scale operations.

The computing device (400) includes a baseboard (402), or “motherboard,” which is a printed circuit board to which a multitude of components or devices can be connected via a system bus or other electrical communication paths. In one illustrative configuration, one or more central processing units (“CPUs”) (404) operate in conjunction with a chipset (406). The CPUs (404) are standard programmable processors that perform arithmetic and logical operations necessary for the operation of the computing device (400).

The CPUs (404) perform operations by transitioning from one discrete, physical state to the next through the manipulation of switching elements that differentiate between and change these states. These basic switching elements can be combined to create more complex logic circuits, including registers, adders-subtractors, arithmetic logic units, floating-point units, and similar components necessary for implementing the clinical rules processor (231) and STMO processors (233) shown in FIG. 2.

The chipset (406) provides an interface between the CPUs (404) and the remainder of the components and devices on the baseboard (402). The chipset (406) can provide an interface to a random access memory (RAM) (408), which serves as the main memory in the computing device (400). The chipset (406) can further provide an interface to a computer-readable storage medium, such as a read-only memory (ROM) (410) or non-volatile RAM, for storing basic routines that help to start the computing device (400) and transfer information between the various components and devices.

The computing device (400) can operate in a networked environment using logical connections to remote computing devices and computer systems through a network, such as connections to cloud storage service providers (300). The chipset (406) can include functionality for providing network connectivity through a Network Interface Controller (NIC) (412), such as a gigabit Ethernet adapter. The NIC (412) can connect the computing device (400) to cloud storage service providers (300) over a network. Multiple NICs (412) can be present in the computing device (400), connecting the computer to different types of networks and remote computer systems.

The computing device (400) can be connected to computer-readable media (418) that provide non-volatile storage. The computer-readable media (418) can store an operating system (420) and programs (422) that implement the functionality described throughout this application. The computer-readable media (418) can be connected to the computing device (400) through a storage controller (414) connected to the chipset (406). The computer-readable media (418) can include one or more physical storage units, which may implement the database system (220) shown in FIG. 2.

The storage controller (414) can interface with the physical storage units through a serial attached SCSI (SAS) interface, a serial advanced technology attachment (SATA) interface, a fiber channel (FC) interface, APIs provided by cloud storage providers, or other type of interface for physically connecting and transferring data between computers and physical storage units. These interfaces enable the storage and retrieval of both medical image data and associated metadata as described in the database system (220) section.

The computing device (400) can store data on the computer-readable media (418) by transforming the physical state of the physical storage units to reflect the information being stored. For example, the computing device (400) can store information to the computer-readable media (418) by issuing instructions through the storage controller (414) to alter the magnetic characteristics of a particular location within a magnetic disk drive unit, the reflective or refractive characteristics of a particular location in an optical storage unit, or the electrical characteristics of a particular capacitor, transistor, or other discrete components in a solid-state storage unit.

The computing device (400) can also include one or more input/output controllers (416) for receiving and processing input from several input devices, such as a keyboard, a mouse, a touchpad, a touch screen, an electronic stylus, or another type of input device. Similarly, an input/output controller (416) can provide output to a display such as a computer monitor, a flat panel display, a digital projector, or another type of output device. These input/output devices can be used by healthcare administrators to interact with the user interface (240) shown in FIG. 2.

When loaded with specific computer-executable instructions, these transform the general-purpose computing device (400) into a specialized machine implementing the described functions of the clinical rules processor (231) and STMO processors (233), and access pattern analyzing processor (235) shown in FIG. 2. These instructions dictate how CPUs (404) transition between states, enabling all operations previously described.

The computer-readable media (418) or other computer-readable storage media are encoded with computer-executable instructions that, when loaded into the computing device (400), transform the device from a general-purpose computing system into a special-purpose computer capable of implementing the clinically intelligent image lifecycle management system described herein. These computer-executable instructions implement the functionality of the interface processor (210), the archive managing processor (230), and the storage interface processor (250) shown in FIG. 2.

### 6. Computer-Integrated Rules Application Examples

### 6.1 DICOM Example: POCUS Image Management

In this example, a POCUS examination is performed in an emergency department using a portable ultrasound device. The portable ultrasound device generates a series of DICOM images and video clips. The DICOM images are transmitted over the healthcare enterprise network to the interface processor (210) of the image lifecycle management system (120). The interface processor (210), implemented on the computing device (400) shown in FIG. 4, receives these images through the Network Interface Controller (412). Upon receipt, the computing device's CPUs (404) process the incoming DICOM data and temporarily store it in the RAM (408) for immediate processing.

The archive managing processor (230), running as a specialized software component on the computing device (400), examines the DICOM headers of the received images. The clinical rules processor (231) extracts key metadata elements, including: Modality (ultrasound), Department (Emergency Department), Study Description (Right Upper Quadrant POCUS), Patient's Age (45 years old), Referring Physician (Dr. Brian Smith), Request Urgency (STAT, i.e., immediately necessary).

The clinical rules processor (231) applies a first set of rules to this metadata and determines that this is a POCUS examination, which typically has high immediate clinical relevance but low long-term reference value. Based on this initial classification, the system assigns the image data to a specific STMO processor (233) that handles emergency ultrasound data.

The designated STMO processor (233) executes a command message that defines: (i) access control permissions, allowing immediate access by emergency department physicians and radiologists, and other members of the care team based on their role; (ii) initial cloud storage tier placement, designating hot tier storage (310) for 14 days to ensure immediate availability during the patient's emergency department stay; (iii) automated lifecycle management, requesting migration to cool tier storage (320) after 14 days, then to cold tier storage (330) after 60 days, and finally to archive tier storage (340) after one year.

The storage interface processor (250), also running on the computing device (400), receives this command message and prepares to implement the storage strategy. It formats an API request to the cloud storage provider (300) via the Network Interface Controller (412). The medical image data, which has been temporarily held in the fast storage medium (221) of the database system (220), is then transferred through the network to the hot tier storage (310) of the cloud storage provider.

Concurrently, the clinical metadata extracted from the DICOM headers is stored separately in the solid-state drive (223) of the database system (220) through operations executed by the CPUs (404) and managed by the storage controller (414).

Sixty days after the initial storage, the STMO processor (233) generates a new command message requesting migration of the POCUS images from the cool tier storage (320) to the cold tier storage (330). The storage interface processor (250) sends an API call to the cloud storage provider (300) that reclassifies the images without physically moving the data, significantly reducing transaction costs associated with retrieval and re-storage operations.

### 6.2 Non-DICOM Example: Wound Care Photography

In this example, a wound care specialist uses a digital camera to photograph a patient's burn wound for documentation and treatment monitoring. The resulting images are high-resolution JPEG files, not DICOM images.

The JPEG images are uploaded from the storage card of the digital camera to the interface processor (210) of the image lifecycle management system (120) using the user interface (240). The interface processor (210) receives the non-DICOM images through the Network Interface Controller (412) of the computing device (400).

Upon receipt, the interface processor (210) recognizes that these are non-DICOM images and employs specialized handling routines. The CPUs (404) process the image, extracting available metadata including: File format (JPEG), Creation date/time (May 1, 2025, 10:15 am), Image dimensions (3840×2160 pixels), Device information (Sony A7 III), and File size (12.4 MB). Since the non-DICOM image lacks standardized medical metadata fields, the interface processor (210) communicates with the hospital's Admission, Discharge, Transfer (ADT) interface to gather additional clinical context: (Patient ID: 123-XXYY), Encounter type (Wound Care Follow-up), Wound type (Diabetic foot ulcer), Wound location (Left foot, plantar surface), Provider (Dr. Brian Smith), and Treatment status (Active treatment, week 3).

The clinical rules processor (231) analyzes this combined metadata and classifies it as a wound care image with ongoing clinical relevance during the active treatment phase. It assigns the image to a specialized STMO processor (233) that handles wound care images. The designated STMO processor (233) executes a command message that defines: (i) access control permissions, allowing access by the wound care team, vascular specialists, and primary care providers, (ii) initial cloud storage tier placement, designating hot tier storage (310) during active treatment, then migrate to cool tier storage (320) for 60 days, followed by cold tier storage (330) for long-term retention.

The storage interface processor (250) implements this strategy by sending appropriate standardized API requests to the cloud storage provider (300). The wound care image is transferred from the fast storage medium (221) to the hot tier storage (310). Based on the clinically intelligent ILM rules associated with the STMO, the STMO processor (233) generates a new command message requesting migration to cool tier storage (320), which is implemented through an API call to reclassify the image data without physical retrieval and re-storage.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter, such that the disclosed subject matter should be recognized as also specifically directed to other embodiments having any other possible combinations. Thus, the foregoing description of specific embodiments of the disclosed subject matter has been presented for illustration and description purposes. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

### LIST OF REFERENCE SIGNS

100—System for storing and managing medical image data
110—Medical imaging network
120—Clinically intelligent image lifecycle management system
130—Cloud storage providers
131—High-performance tier (hot tier storage)
133—Intermediate tier (cool tier storage)
135—Archival tier (cold tier storage)
210—Interface processor
220—Database system
221—Fast storage medium
223—Solid-state drive
230—Archive managing processor
231—Clinical rules processor
233—Storage Management Organization (STMO) processors
235—Access pattern analyzing processor
240—User interface
250—Storage interface processor
300—Cloud storage service provider architecture
310—Hot tier storage
320—Cool tier storage
330—Cold tier storage
340—Archive tier storage
350—Application Programming Interface (API)
400—Computing device
402—Baseboard (motherboard)
404—Central processing units (CPUs)
406—Chipset
408—Random access memory (RAM)
410—Read-only memory (ROM)
412—Network Interface Controller (NIC)
414—Storage controller
416—Input/output controllers
418—Computer-readable media
420—Operating system
422—Programs

1. An image lifecycle management system connected to receive messages over a healthcare enterprise network, the system comprising:
a) an interface processor for receiving and forwarding data comprising medical image data and associated clinical metadata;
b) a database system comprising a fast storage medium for temporary storage and a solid-state drive for long-term storage of metadata;
c) an archive managing processor configured to:
temporarily store the data forwarded from the interface processor in the fast storage medium;
analyze the metadata based on clinical intelligent rules and classify the image data based on the analyzed metadata;
assign the classified image data to a plurality of designated Storage Management Organization (STMO) processors, respectively,
wherein each STMO processor executes a command message on its respective image data, defining:
(i) access control permissions;
(ii) initial cloud storage tier placement of its respective data; and
(iii) automated lifecycle management;
send the respective command message executed by each STMO processor to a cloud storage service provider; AND
d) a storage interface processor for connecting to the cloud storage services via an application programming interface (API),
wherein the storage interface processor uses a network to transfer the respective image data stored in the fast storage medium to the respective cloud storage tier,
wherein a plurality of storage devices are organized in tiers, and each tier has cost and performance characteristics that differ from other tiers; and
wherein the transferring is responsive to the respective command message executed by each STMO.

2. The image lifecycle management system of claim 1, wherein the archive managing processor is further configured to separate the data into the medical image data and the clinical metadata, wherein the clinical metadata is transferred and stored in the solid-state drive.

3. The image lifecycle management system of claim 1, wherein the data comprising medical image data and associated clinical metadata is received from medical imaging devices on the healthcare enterprise network.

4. The image lifecycle management system of claim 1, wherein the access control permissions determine which users and systems can view, modify, or delete the respective classified data of each STMO processor.

5. The image lifecycle management system of claim 1, wherein the initial cloud storage tier placement is based on anticipated clinical relevance and access frequency as determined by the clinically intelligent rules.

6. The image lifecycle management system of claim 1, wherein the automated lifecycle management determines migration of respective medical image data between cloud storage tiers as clinical relevance changes over time in accordance with organizational image retention policies.

7. The image lifecycle management system of claim 1, wherein the clinical rules include parameters related to one or more of:
Accession Number, Acquisition Date, Acquisition Time, Body Part Content Date, Content, Time Ethnic Group, Image Number, Image Type, Institution Name, Institutional Department Name, Issuer of Patient ID, Laterality, Modality, Patient's Age, Patient's Birth Date/Time, Patient's First Name, Patient's Last Name, Patient's Sex, Patient's Size, Patient's Weight, Patient ID, Procedure Code Value, Referring Physician's First Name, Referring Physician's Last Name, Requesting Physician's First Name, Requesting Physician's Last Name, Requesting Service, SOP Class UID, SOP Instance UID, Scheduled Study Start Date/Time, Series Description, Series Number, Series UID, Slice Thickness, Station Name, Study Completion Date, Study Completion Time, Study Date/ Time, Study Description, and Study ID.

8. The image lifecycle management system of claim 1, wherein the archive managing processor is further configured to:
(a) request to store the image data in the cloud storage tier responsive to the respective message STMO when the respective message is related to the storage category;
(b) request to retrieve the image data from the cloud storage tier responsive to the respective message STMO when the request category is related to the retrieve category; and
(c) request to coordinate with the cloud storage service provider to migrate the image data between cloud storage tiers responsive to the respective message STMO when the request category is related to the migrate category.

9. The image lifecycle management system of claim 1, wherein the system manages both Digital Imaging and Communications in Medicine (DICOM) and non-DICOM data.

10. The image lifecycle management system of claim 9, wherein the non-DICOM data comprises at least one of Joint Photographic Experts Group (JPEG) images, Portable Network Graphics (PNG) images, Tagged Image File Format (TIFF) images, Portable Document Format (PDF) files, Moving Picture Experts Group (MPEG) videos, and Audio Video Interleave (AVI) videos.

11. The image lifecycle management system of claim 6, wherein the migration of image data between storage tiers is performed using Application Programming Interface (API) calls to reclassify the image data within the cloud storage service provider in place without physically retrieving and re-storing the image data.

12. The image lifecycle management system of claim 1, wherein the system further comprises a computing device comprising at least one central processing unit (CPU), a chipset, random access memory (RAM), a read-only memory (ROM), and a network interface controller, wherein the computing device executes computer-executable instructions implementing the archive managing processor and the storage interface processor to transfer image data to the cloud storage services.

13. A method for managing lifecycle of medical images across storage tiers of a cloud storage service provider, comprising:
a) receiving medical image data and associated metadata from an imaging device;
b) analyzing the metadata using a set of clinically intelligent rules to determine an initial storage tier of the cloud storage service provider for the image data;
c) storing the image data in the determined cloud storage tier;
d) storing the metadata in a local database separate from the image data;
e) periodically evaluating the stored image data against the set of clinically intelligent rules to determine whether it should be migrated to a different storage tier; and
f) when migration to a different tier is indicated, reclassifying the image data within the cloud storage service provider using API calls without physically moving the image data.

14. The method of claim 13, wherein the step of analyzing the metadata includes determining category of the image data based on parameters related to one or more of:
Accession Number, Acquisition Date, Acquisition Time, Body Part Content Date, Content, Time Ethnic Group, Image Number, Image Type, Institution Name, Institutional Department Name, Issuer of Patient ID, Laterality, Modality, Patient's Age, Patient's Birth Date/Time, Patient's First Name, Patient's Last Name, Patient's Sex, Patient's Size, Patient's Weight, Patient ID, Procedure Code Value, Referring Physician's First Name, Referring Physician's Last Name, Requesting Physician's First Name, Requesting Physician's Last Name, Requesting Service, SOP Class UID, SOP Instance UID, Scheduled Study Start Date/Time, Series Description, Series Number, Series UID, Slice Thickness, Station Name, Study Completion Date/Time, Study Date/Time, Study Description, and Study ID.

15. The method of claim 13, further comprising assigning the image data with different lifecycle parameters across storage tiers based on the set of clinically intelligent rules.

16. The method of claim 13, wherein the method is applied to both Digital Imaging and Communications in Medicine (DICOM) and non-DICOM data.

17. The method of claim 16, wherein the non-DICOM data comprises at least one of Joint Photographic Experts Group (JPEG) images, Portable Network Graphics (PNG) images, Tagged Image File Format (TIFF) images, Portable Document Format (PDF) files, Moving Picture Experts Group (MPEG) videos, and Audio Video Interleave (AVI) videos.
